(19) United States
(12) Patent Application Publication
(10) Pub. No.: US 2009/0142411 A1
(43) Pub. Date: Jun. 4, 2009

(54) Composition and Device Comprising an Inorganic Component (Metal Compound) for Coagulation of Protein-Containing Fluids

(75) Inventors: Joanna Buckley, London (GB); Bryan Greener, London (GB); Emma Woodmansey, London (GB)

Correspondence Address: DIANA HOUSTON;SMITH & NEPHEW, INC., 1450 BROOKS ROAD, MEMPHIS, TN, 38116, US

(73) Assignee: SMITH & NEPHEW, PLC, London (GB)

(21) Appl. No.: 11/814,143

(22) PCT Filed: Jan. 17, 2006
(86) PCT No.: PCT/GB06/00133

§ 371 Date: Aug. 28, 2007

(30) Foreign Application Priority Data
Jan. 18, 2005 (GB) 0500898.2

(51) Int. Cl. A61K 33/24; A61K 33/38; C01G 7/00; C01B 9/02; C01B 9/04; C01B 9/06

(52) U.S. Cl. 424/618; 424/649; 423/491; 423/604; 423/472

(57) ABSTRACT

A device or composition comprising an agent for the coagulation of protein-containing fluids, wherein the agent comprises an inorganic component which is soluble in protein-containing fluids. Use of such a device or composition for the coagulation of protein-containing fluids. A coagulum of protein-containing fluid produced using such a device or composition.

This invention relates to a device for the control of bodily fluids, for example a coagulant and/or sealant device based upon a biomolecular reactive agent which acts on protein-contained in the bodily fluid to effect coagulation of the protein. Examples of the bodily fluid include accumulated reservoirs of proteinaceous bodily fluids such as whole blood, serum, plasma, interstitial fluid, synovial fluid, lymphatic fluid, and wound material, such as wound exudate. The latter may be treated with the present device in the course of management of post surgical, acute or chronic wounds for the promotion of wound healing.

It also relates to a composition for the control of bodily fluids, for example a coagulant and/or sealant composition based upon a biomolecular reactive agent which acts on protein-contained in the bodily fluid to effect coagulation of the protein. Examples of the bodily fluid include accumulated reservoirs of proteinaceous bodily fluids as noted above Examples of the composition include a composition of gold and/or gold compounds.

It also relates to the use of such a coagulant and/or sealant agent, composition or device, based upon a biomolecular reactive agent which acts on protein-contained in bodily fluids, to control the bodily fluid. It also relates to a coagulum of protein-containing fluid produced using such a coagulant and/or sealant agent, composition or device.

Medically, the coagulation of bodily fluids is important in the management of acute haemorrhages and acute trauma injuries to the circulatory system; emergency interventions in these situations are critically important to saving life. Accumulated reservoirs of proteinaceous material are a ready source of food for pathogens including bacteria.

In wound care, the management of post-surgical or acute or chronic wound exudates is both inconvenient for the patient and career and expensive to administer. In the use of a coagulant and/or sealant agent, composition or device in the treatment and management of post surgical, acute or chronic wounds for the promotion of wound healing, not only is the flow of bodily fluids controlled by a reactive agent which acts on protein-contained in the fluids present during bleeding, but a coagulum of protein is produced using such a coagulant and/or sealant agent, composition or device, which acts as an antibacterial barrier, and provides a moist wound environment

In commercial medical applications, biological coagulant and sealant systems are frequently based upon fibrinogen-thrombin-compositions, mixed on application, to form a coagulum or clot. This approach is relatively expensive and requires refrigerated storage. It is also most appropriate for use in the presence of other components of the coagulation cascade (e.g. platelets) present during bleeding.

A less expensive, broad-spectrum coagulant of protein-containing solutions with a long shelf-life is commercially desirable.

An object of this invention is the provision of a device or composition comprising an agent for the coagulation of bodily fluids, particularly whole blood or exudates at the site of trauma, including wounding, and especially a less expensive, broad-spectrum coagulant of protein-containing solutions with a long shelf-life.

An object of this invention is the provision of a device or composition comprising a coagulant of protein-containing fluids that is effective in the promotion of wound healing.

A further object of this invention is the provision of an antibacterial coagulum of protein-containing solution. This is particularly appropriate for applications exposed to potential for bacterial contamination including surgical sites and surface wounds including acute and chronic wounds.

A further object of this invention is the provision of a device or composition comprising a coagulum of protein-containing solution that provides a moist environment for the promotion of wound healing.

A further object of this invention is the provision of a device or composition comprising a coagulum of protein-containing solution that provides an antibacterial, moist environment for the promotion of wound healing.

A further object of this invention is the provision of a device or composition comprising a coagulum of protein-containing solution that provides an antibacterial barrier and moist environment for the promotion of wound healing.

According to a first aspect of the present invention there is provided a device comprising an agent for the coagulation of protein-containing fluids, wherein the agent comprises an inorganic component which is soluble in protein-containing fluids.

According to a second aspect of the present invention there is provided a composition comprising an agent for the coagulation of protein-containing fluids, wherein the agent comprises an inorganic component which is soluble in protein-containing fluids.

According to a third aspect of the present invention there is provided a use of a device or composition according to the first or second aspects of the present invention for the coagulation of protein-containing fluids.

According to a fourth aspect of the present invention there is provided a coagulum of protein-containing fluid produced using a device or composition according to the first or second aspects of the present invention.

According to a fifth aspect of the present invention there is provided a device or composition comprising a coagulum of protein-containing fluids according to the fourth aspect of the present invention.

The protein-containing fluids may comprise bodily fluids, such as whole blood, serum, plasma, interstitial fluid, synovial fluid, lymphatic fluid, wound exudates, semen, saliva, spinal-cord fluid and ocular fluid.

Preferably, the inorganic component comprises a metal. Preferably, the inorganic component comprises a metal compound. Preferably, the metal is gold.

The metal compound may be metal chloride, metal bromide, metal iodide, metal oxide or metal hydroxide.

Suitable metal compounds include those readily soluble in aqueous media or solvent mixtures compatible with aqueous media, or partially soluble in aqueous media or solvent mixtures compatible with aqueous media, or sparingly soluble in aqueous media or solvent mixtures compatible with aqueous media.

We have discovered that the presence of a sufficient concentration of soluble gold species in protein-containing solutions results in-coagulation and the formation of a device or composition comprising a heterogeneous mass that can grossly retain its set dimensions unsupported. Examples of such proteinaceous fluids include proteinaceous bodily fluids as noted above, including: bovine serum albumin solution, gelatin, foetal calf serum, whole human blood, acute wound fluid and chronic wound fluid

It is envisaged that other transition metals would be suitable agents for the coagulation of protein-containing fluids.

Gold compounds are known antibacterial agents and have a similar potency to silver compounds in in vitro tests. Silver compounds on exposure to bodily fluid (pH 7-8) however are rapidly precipitated to form insoluble, and therefore inactive, silver chloride.

This situation can be avoided, to a limited extent, by raising or lowering the local pH, which facilitates the formation of soluble hydrated and/or hydroxylated silver species.

In medical device applications, silver oxide-based dressings generate elevated pH in their local environment, thus enabling solubilisation of significant concentrations of silver species.

Generation of a device or composition comprising a non-neutral pH (usually alkaline from oxide species) is therefore a pre-requisite to silver-based medical device activity.

The generation, even locally, of such a pH is however not generally considered to be biologically advantageous.

Gold is distinct from silver in that its salts, e.g. the chloride tend to be soluble in biological fluid, and therefore gold species precursors, such as gold oxide, readily form soluble gold compounds at neutral (or any other) pH.

Gold devices, therefore, can be employed for their intended use, including antibacterial activity, without the additional need for potentially detrimental environmental pH change.

The mechanism of action of gold compounds is significantly advantageous in-comparison to silver-based counterparts

Gold compounds are also known and have been medically investigated and exploited for their anti-inflammatory properties in degenerative conditions such as rheumatoid arthritis.

The devices and composition for the control of bodily fluids of the present invention thus also have an anti-inflammatory effect.

A further object of this invention is the provision of a device or composition comprising a coagulant for a protein-containing solution that provides an anti-inflammatory effect.

Some of the devices and compositions of this invention undergo colour change in use and thus may self-indicate wear-time.

A further object of this invention is the provision of a device or composition comprising a coagulant of protein-containing solution that self-indicates wear-time by colour change.

An object of this invention is the provision of a device or composition that provides an antibacterial barrier, moist environment and anti-inflammatory effect for the promotion of wound healing

A further object of this invention is the provision of a device or composition comprising a coagulant of a protein-containing solution that provides an antibacterial barrier, moist environment and anti-inflammatory effect and that self-indicates wear-time by colour change.

Suitable gold compounds include those

readily soluble in aqueous media or solvent mixtures compatible with aqueous media, or
partially soluble in aqueous media or solvent mixtures compatible with aqueous media, or
sparingly soluble in aqueous media or solvent mixtures compatible with aqueous media.

Examples of readily-soluble gold compounds include gold (III) bromide, gold (III) iodide, gold (I) iodide, gold (III) chloride [AuCl₃] and its hydrochloric acid salt, chloroauric acid [H⁺AuCl₄].

Examples of partially-soluble gold compounds include gold (III) oxide [Au₂O₃] and gold (III) hydroxide [Au(OH)₃].

The gold compounds generate solvated gold species in solution. These species may be the same or different from the precursor gold compound, for example [Au(OH)₃Cl]⁻ is a commonly occurring product of gold compound dissolution.

These species may also be atomic cluster species, including colloidal species.

The composition for the control of bodily fluids of the present invention may comprise a coagulant for a protein-containing solution which consists of one or more gold compounds, or the compound(s) may be in admixture with other materials, for example gold, other metals, other metallic compounds, organic compound (such as pharmacologically active compounds, or proteins or other complex synthetic or natural materials.

Mixtures must be such that the coagulative properties of the mixture are retained, and preferably the antibacterial properties of the mixture are also retained.

Examples of mixed compositions include mixtures of gold compounds with gold, silver and/or silver compounds, preferably mixtures of gold, gold oxides and silver and silver oxides, and more preferably mixtures of gold (III) oxide and silver (I/III) oxide. Most preferred composition for the control of bodily fluids of the present invention are those which in use cause a physiologically acceptable pH in the locality of application.

Protein-containing solutions include those of individual proteins or mixtures of an infinite number of protein-compounds. Protein-containing solutions include bodily fluids such as whole blood, serum, plasma, interstitial fluid, synovial fluid, lymphatic fluid, wound exudates, semen, saliva, spinal-cord fluid or ocular fluid for example or mixtures of these. These solutions are most often the bodily fluids which it is wished to control. However, it may be desired to introduce or generate a coagulum of other protein-containing fluid using such a coagulant and/or sealant agent, composition or device in situ, e.g. to exclude pathogens including bacteria.

Protein-containing solutions thus include those arising from any protein-based biological system, including animal species including mammals, amphibians, birds, fish or insects; or plants. Such protein-containing solutions may contain recombinant or synthetically modified proteins. All such proteins can be in a native or denatured conformation.

The gold compounds can be present in any form of device or composition comprising a coagulant for a protein-containing solution that is known to be suitable for such an effect to those skilled in the art. Such compositions include fluids, such as solutions, e.g. AuCl₃ (aq), or emulsions, e.g. Au₂O₃ suspended in mineral oil and water mixtures, or colloidal suspensions, e.g. gold cluster species in aqueous solution, or gels, e.g. gold cluster species dispersed in a hydrogel such as hydrated carboxymethyl cellulose, or solids, e.g. Gold(III) oxide, gold(III) hydroxide, deposited colloidal gold particles or dispersions in hydrophilic plastic films e.g. polyurethane films, or dispersions in hydrophilic foams, e.g. polyurethane foams.

Such devices include: film dressings for the protection of fragile biological surfaces and the occlusion of moisture, foams for the management of biological exudates and hydrocolloid gels for the management of tissue hydration.

Such devices preferably include devices for the management of infection, in particular bacterial infection, including the treatment of antibiotic-resistant bacterial strains (e.g. MRSA).

Such devices also include formats for the treatment of medical waste and the contents of medical facilities, for example exudates drains, operating surfaces and surfaces harbouring bacteria.

Such devices may comprise one or more layers of any of the foregoing compositions applied to a substrate by any deposition technique known to those skilled in the art.

The composition or device and its method of preparation is preferably compatible with the stability (e.g. thermal stability) of the active species present.

In the case of gold(III) oxide, processing temperatures should not exceed 300° C.

One suitable method of preparation of the composition is direct mixing with a carrier at ambient temperature.

The delivery system can be applied directly to the site to be treated or may be used remotely as part of a remote patient management system.

In this format, the gold compound can be localised for the sequestration of proteins (by coagulation) from protein-containing solutions, including bodily fluids.

### EXAMPLES

### Example 1

Gold (III) oxide powder (10 mg), was added to a solution of bovine serum albumin (100 μl, 40 mg/ml) made up in phosphate-buffered saline (pH 7.4).

The oxide powder was allowed to settle to the bottom of the vessel (0.5 ml capacity Eppendorf tube) and was left undisturbed for 2 hours.

After this time, a coagulated, opaque mass of several millimetres thickness was formed in-contact with the gold (III) oxide powder.

The vessel could be inverted without disturbance of the opaque mass.

### Example 2

Gold (III) oxide powder (10 mg), was added to heat-inactivated foetal calf serum (100 μl).

The oxide powder was allowed to settle to the bottom of the vessel (0.5 ml capacity Eppendorf tube) and was left undisturbed for 2 hours. After this time, a coagulated, opaque mass of several millimetres thickness was formed in-contact with the gold (III) oxide powder.

The vessel could be inverted without disturbance of the opaque mass.

### Example 3

Gold (III) oxide powder (10 mg), was added to chronic wound fluid (100 μl). The oxide powder was allowed to settle to the bottom of the vessel (0.5 ml capacity Eppendorf tube) and was left undisturbed for 2 hours.

After this time, a coagulated, opaque mass of several millimetres thickness was formed in-contact with the gold (III) oxide powder.

The vessel could be inverted without disturbance of the opaque mass. Overnight, the entire fluid volume had coagulated.

### Example 4

A 1 μl aliquot of a solution of gold (III) chloride (350 mg/ml) in phosphate buffered saline, pH 7.4, was carefully added to the bottom of a vessel (0.5 ml capacity Eppendorf tube) containing a solution of bovine serum albumin (100 μl, 40 mg/ml) made up in phosphate-buffered saline (pH 7.4).

The vessel was left undisturbed for 30 minutes.

During this time, a coagulated, opaque mass of several millimetres thickness was formed. The vessel could be inverted without disturbance of the opaque mass.

### Example 5

A 2 μl aliquot of a solution of gold (III) chloride (350 mg/ml) in phosphate buffered saline, pH 7.4, was carefully added to the bottom of a vessel (0.5 ml capacity Eppendorf tube) containing a solution of bovine serum albumin (100 μl, 40 mg/ml) made up in phosphate-buffered saline (pH 7.4). The vessel was left undisturbed for 30 minutes. During this time, a coagulated, opaque mass of several millimetres thickness was formed. The vessel could be inverted without disturbance of the opaque mass.

### Example 6

A 3 μl aliquot of a solution of gold (III) chloride (350 mg/ml) in phosphate buffered saline, pH 7.4, was carefully added to the bottom of a vessel (0.5 ml capacity Eppendorf tube) containing a solution of bovine serum albumin (100 μl, 40 mg/ml) made up in phosphate-buffered saline (pH 7.4). The vessel was left undisturbed for 30 minutes.

During this time, a coagulated, opaque mass of several millimetres thickness was formed. The vessel could be inverted without disturbance of the opaque mass.

### Example 7

A 4 μl aliquot of a solution of gold (III) chloride (350 mg/ml) in phosphate buffered saline, pH 7.4, was carefully added to the bottom of a vessel (0.5 ml capacity Eppendorf tube) containing a solution of bovine serum albumin (100 μl, 40 mg/ml) made up in phosphate-buffered saline (pH 7.4). The vessel was left undisturbed for 30 minutes.

During this time, a coagulated, opaque mass of several millimetres thickness was formed. The vessel could be inverted without disturbance of the opaque mass.

### Example 8

A 5 μl aliquot of a solution of gold (III) chloride (350 mg/ml) in phosphate buffered saline, pH 7.4, was carefully added to the bottom of a vessel (0.5 ml capacity Eppendorf tube) containing a solution of bovine serum albumin (100 μl, 40 mg/ml) made up in phosphate-buffered saline (pH 7.4). The vessel was left undisturbed for 30 minutes.

During this time, a coagulated, opaque mass of several millimetres thickness was formed. The vessel could be inverted without disturbance of the opaque mass.

### Example 9

A digital image of the results of experiments in Examples 4-8, with the addition of a device or composition comprising a blank control, was recorded. It could be seen that a linear increase in the quantity of gold present resulted in a comparable linear increase in the depth of coagulated mass.

### Example 10

Examples 4-9 were repeated with 100 μl heat-inactivated foetal calf serum in place of the bovine serum albumin solution. The results were the same.

### Example 11

Examples 4-9 were repeated with 100 μl chronic wound fluid in place of the bovine serum albumin solution. The results were the same.

### Example 12

Examples 4-9 were repeated with 100 μl whole human blood in place of the bovine serum albumin solution. The results were the same.

### Example 13

A 5 gi aliquot of a solution of gold (III) chloride (350 mg/ml) in phosphate buffered saline, pH 7.4, was carefully added to the bottom of a vessel (0.5 ml capacity Eppendorf tube) containing whole human saliva (100 μl). The vessel was left undisturbed for 30 minutes. During this time, a lightly coagulated, opaque mass of several millimetres thickness was formed.

### Example 14

A 5 μl aliquot of a solution of gold (III) chloride (350 mg/ml) in phosphate buffered saline, pH 7.4, was carefully added to the bottom of a vessel (0.5 ml capacity Eppendorf tube) containing 100 μl gelatine solution (40 mg/ml) made up in phosphate buffered saline.

Immediately, a fibrous precipitate was formed that could be drawn in viscous strands out of the vessel.

### Example 15

A 5 μl aliquot of a solution of gold (III) chloride (350 mg/ml) in phosphate buffered saline, pH 7.4, was carefully added to the bottom of a vessel (1.0 ml capacity syringe with tip removed) containing a solution of bovine serum albumin (100 gi, 40 mg/ml) made up in phosphate-buffered saline (pH 7.4). The vessel was left undisturbed for 24 h.

During this time, the fluid coagulated. The syringe plunger was used to expel the coagulated plug. The plug retained its dimensions unsupported.

### Example 16

A 5 μl aliquot of a solution of gold (III) chloride (350 mg/ml) in phosphate buffered saline, pH 7.4, was carefully added to the bottom of a vessel (1.0 ml capacity syringe with tip removed) containing heat-inactivated foetal calf serum. The vessel was left undisturbed for 24 h.

During this time, the fluid coagulated. The syringe plunger was used to expel the coagulated plug. The plug retained its dimensions unsupported.

### Example 17

A 5 μl aliquot of a solution of gold (III) chloride (350 mg/ml) in phosphate buffered saline, pH 7.4, was carefully added to the bottom of a vessel (1.0 ml capacity syringe with tip removed) containing chronic wound fluid. The vessel was left undisturbed for 24 h. During this time, the fluid coagulated.

The syringe plunger was used to expel the coagulated plug. The plug retained its dimensions unsupported.

### Example 18

Pseudomonas aeruginosa NCIMB 8626 and Staphylococcus aureus NCTC 10788 were harvested. Serial 1:10 dilutions were performed to give a final concentration of 10⁸ bacteria/ml. Further dilutions were made for an inoculum count, down to 10⁻⁸ bacteria/ml, with the number of bacteria/ml determined using the pour plate method.

Two large assay plates were then set up and 140 ml of Mueller-Hinton agar was added evenly to the large assay plates and allowed to dry (15 minutes). A further 140 ml of agar was seeded with the corresponding test organism and poured over the previous agar layer. Once the agar had set (15 minutes), the plate was dried at 37° C. for 30 minutes with the lid removed.

In triplicate, the plugs resulting from Examples 14-16 were placed, circular face down onto each plate. The plates were then sealed and incubated at 37° C. for 24 hours. The diameter of the bacterial zone cleared was measured using a Vernier calliper gauge:

### Total Zone Measurements (in mm)

| | | Repetitions | | | |
|---|---|---|---|---|---|
| | | 1 | 2 | 3 | MEAN |
| P. aeruginosa NCIMB | albumin | 10.5 | 9.8 | 9.6 | 10.0 |
| 8626 | foetal calf | 13.6 | 11.8 | 11.5 | 12.3 |
| | serum | | | | |
| | chronic wound | 14.5 | 14.0 | 14.1 | 14.2 |
| | fluid | | | | |
| S. aureus NCTC 10788 | albumin | 10.4 | 10.6 | 12.4 | 11.1 |
| | foetal calf | 12.7 | 13.5 | 13.2 | 13.1 |
| | serum | | | | |
| | chronic wound | 15.3 | 14.8 | 14.7 | 14.9 |
| | fluid | | | | |

In each case, the coagulated plug generated a zone of bacterial clearance.

### Example 19

Standard cotton-bud swabs (x8) were immersed in gold(III) chloride solution (35 mg/ml) made up in distilled water. The swab was removed and allowed to air dry at room temperature, resulting in a gold(III) chloride coating. Controls (x8) were prepared in the absence of gold(III) chloride. The swabs were immersed individually in 400 ul chronic wound fluid situated in a 2 ml capacity Eppendorf, the swabs were left in place for 14 hours. After this time, the swabs were removed and the Eppendorfs were capped and inverted. For the gold-swabbed set, fluid was immobilised by coagulation. For the control set, fluid flowed as normal. The gold swabs inhibit the movement of wound fluid by coagulation.

### Example 20

### Gold(III) Oxide Impregnated Hydrophilic Polyurethane Film

Hydrophilic polyurethane HPU25 (Smith & Nephew Medical Limited) was dissolved in minimum volume tetrahydrofuran, to a viscosity suitable for film-spreading. 100 mg of gold(III) oxide powder (Aldrich Chemical Co.) was dispersed in the solvated polyurethane by mixing.

The resulting mass was spread as a film of approximately 100 micron thickness, residual solvent was allowed to evaporate. The resulting film was suitably robust for medical device applications and was transparent pink in-colour, indicating the presence of colloidal gold species.

A gold-free blank film was prepared by the same method.

### Example 21

2 cm squares of the films prepared in Example 20 were tested for antimicrobial activity:

Pseudomonas aeruginosa NCIMB 8626 and Staphylococcus aureus NCTC 10788 were harvested. Serial 1:10 dilutions were performed to give a final concentration of 10⁸ bacteria/ml. Further dilutions were made for an inoculum count, down to 10⁻⁸ bacteria/ml, with the number of bacteria/ml determined using the pour plate method.

Two large assay plates were then set up and 140 ml of Mueller-Hinton agar was added evenly to the large assay plates and allowed to dry (15 minutes).

A further 140 ml of agar was seeded with the corresponding test organism and poured over the previous agar layer. Once the agar had set (15 minutes), the plate was dried at 37° C. for 30 minutes with the lid removed.

In triplicate, the films prepared in Example 20 were placed onto each plate. The plates were then sealed and incubated at 37° C. for 24 hours. The size of the bacterial zone cleared was measured using a Vernier calliper gauge, triplicates were averaged:

| | HPU25 | HPU25 film + Gold(III) |
|---|---|---|
| Average zone size | film | oxide |
| P. aeruginosa | 0.0 mm | 0.56 mm |
| S. aureus | 0.0 mm | 0.71 mm |

During the 24 hour test period, the initially pink coloured films had turned yellow, indicating the dissociation of colloidal gold species and providing a simple indicator of device expiry.

### Example 22

### Gold(III) Oxide Impregnated Hydrogel

100 mg of gold(III) oxide powder (Aldrich Chemical Co.) was dispersed in 50 g of IntraSite Gel (Smith & Nephew Medical Ltd.). The resulting mixture was allowed to stand for 24 h. The resulting gel did not differ mechanically from the initial gel, but was transparent pink in-colour, indicating the presence of colloidal gold species.

A gold-free blank film was prepared by the same method.

### Example 23

### The Hydrogels Prepared in Example 22 were Tested for Antimicrobial Activity

Pseudomonas aeruginosa NCIMB 8626 and Staphylococcus aureus NCTC 10788 were harvested. Serial 1:10 dilutions were performed to give a final concentration of 10⁸ bacteria/ml.

Further dilutions were made for an inoculum count, down to 10⁻⁸ bacteria/ml, with the number of bacteria/ml determined using the pour plate method.

Two large assay plates were then set up and 140 ml of Mueller-Hinton agar was added evenly to the large assay plates and allowed to dry (15 minutes). A further 140 ml of agar was seeded with the corresponding test organism and poured over the previous agar layer. Once the agar had set (15 minutes), the plate was dried at 37° C. for 30 minutes with the lid removed. 8 mm plugs were removed from the plate by biopsy punch.

In triplicate, 200 μl of gels prepared in Example 22 were placed onto each plug hole by 1 ml capacity syringe. The plates were then sealed and incubated at 37° C. for 24 hours. The size of the bacterial zone cleared was measured using a Vernier calliper gauge, triplicates were averaged:

| | IntraSite | IntraSite + Gold(III) |
|---|---|---|
| Average zone size | gel | oxide gel |
| P. aeruginosa | 0.0 mm | 4.4 mm |
| S. aureus | 0.0 mm | 4.8 mm |

### Example 24

Gold (III) oxide powder (10 mg) and silver (I) oxide powder (1 mg) were added to a solution of bovine serum albumin (100 μl, 40 mg/ml) made up in phosphate-buffered saline (pH 7.4).

The oxide powder was allowed to settle to the bottom of the vessel (0.5 ml capacity Eppendorf tube) and was left undisturbed for 2 hours.

After this time, a coagulated, opaque mass of several millimetres thickness was formed in contact with the oxide powder.

It has been found that silver oxides such as silver (I) oxide dissolve significantly in protein-containing fluid and the silver hydroxide species so formed serve to enhance the rate of dissolution of the gold (III) oxide. This demonstrates the utility of applying a percentage of silver oxide to dictate the rate of dissolution of gold (III) oxide into protein-containing solutions.

1. A composition comprising an agent for the coagulation of protein-containing fluids, wherein the agent comprises an inorganic component which is soluble in protein-containing fluids.

2. A composition according to claim 1, wherein the protein-containing fluids comprise bodily fluids.

3. A composition according to claim 1, wherein the inorganic component comprises a metal.

4. A composition according to claim 1, wherein the inorganic component comprises a metal compound.

5. A composition according to claim 3, wherein the metal is gold.

6. A composition according to claim 4, wherein the metal compound is selected from the group consisting of metal chloride, metal bromide, metal iodide, metal oxide and metal hydroxide.

7. A composition according to claim 4, wherein the metal compound is readily soluble in aqueous media or solvent mixtures compatible with aqueous media, or partially soluble in aqueous media or solvent mixtures compatible with aqueous media, or sparingly soluble in aqueous media or solvent mixtures compatible with aqueous media.

8. A composition according to claim 5, wherein the gold is selected from the group consisting of gold (III) bromide, gold (III) iodide, gold (I) iodide, gold (III) chloride, chloroauric acid, gold (III) oxide and gold (III) hydroxide.

9. A composition according to claim 8, wherein the gold generates solvated gold species in solution.

10. A composition according to claim 9, wherein the solvated gold species is an atomic cluster species.

11. A device of composition according to claim 9, wherein the solvated gold species is a colloidal species.

12. A composition according to claim 5, comprising a plurality of gold compounds.

13. A composition according to claim 5, wherein the gold is in admixture with a material selected from the group consisting of other metals, other metallic compounds, and organic compounds.

14. A composition according to claim 13, wherein the organic compound is selected from the group consisting of pharmacologically active compounds, proteins, polymers, other complex synthetic materials, and other complex natural materials.

15. A composition according to claim 5, wherein the gold is mixed with silver and/or silver compounds.

16. A composition according to claim 5, wherein the composition comprises a mixture of gold, gold oxides, silver and silver oxides.

17. A composition according to claim 16, wherein the silver oxides are silver (I) oxide, silver (I, III) oxide, silver (II, III) oxide or silver (III) oxide.

18. A composition according to claim 5, wherein the composition comprises a mixture of gold (III) oxide and silver (I) oxide.

19. A composition according to claim 5, wherein the composition comprises a mixture of gold (III) oxide and silver (I, III) oxide.

20. A composition according to g claim 1, wherein the protein-containing fluids comprise individual proteins.

21. A composition according to claim 1, wherein the protein-containing fluids comprise a plurality of proteins.

22. A composition according to claim 1, wherein the protein-containing fluids are selected from the group consisting of whole blood, serum, plasma, interstitial fluid, synovial fluid, lymphatic fluid, wound exudates, semen, saliva, spinal-cord fluid and ocular fluid.

23. A composition according to claim 1, wherein the composition provides an antibacterial barrier.

24. A composition according to claim 1, wherein the device or composition provides a moist environment.

25. A composition according to claim 1, wherein the composition provides an anti-inflammatory effect.

26. A composition according to claim 1, wherein the composition self-indicates wear-time by color change.

27. (canceled)

28. (canceled)

29. (canceled)

30. A composition according to claim 1, wherein the composition is a fluid, gel or solid.

31. (canceled)

32. (canceled)

33. (canceled)

34. (canceled)

35. (canceled)

36. (canceled)

37. (canceled)

38. (canceled)
